(19) United States
(12) Patent Application Publication
(10) Pub. No.: US 2025/0018070 A1
(43) Pub. Date: Jan. 16, 2025

(54) UV STERILIZATION SYSTEM AND MONITORING METHOD

(71) Applicant: WISTRON CORPORATION, New Taipei City (TW)

(72) Inventors: Jo Ying HSUEH, New Taipei City (TW); Chao Hsu CHEN, New Taipei City (TW); Chien En PENG, New Taipei City (TW)

(73) Assignee: WISTRON CORPORATION, New Taipei City (TW)

(21) Appl. No.: 18/426,599

(22) Filed: Jan. 30, 2024

(30) Foreign Application Priority Data
Jul. 13, 2023 (TW) 112126276

(51) Int. Cl. A61L 2/10

(52) U.S. Cl. CPC A61L 2/10; A61L 2202/11; A61L 2202/14; A61L 2202/16

(57) ABSTRACT

An ultraviolet (UV) sterilization system and monitoring method are applied to detect the working state of each UV lamp tube in an autonomous mobile robot. The monitoring method for UV sterilization includes enabling the autonomous mobile robot to move at a travel speed, driving the UV lamp to illuminate an UV, fetching an inductive voltage for each UV lamp, generating a report according to the inductive voltages for each UV lamp, and sending the report to a monitor server via the network from the autonomous mobile robot. Therefore, the monitoring server can check and record the working state of each UV lamp according to the report, and further the monitoring server will transmit a notification message to a client when the working state of any UV lamp is abnormal.

### CROSS-REFERENCE TO RELATED APPLICATION

This application claims the priority and benefit of Taiwan Patent Application No. 112126276, filed on Jul. 13, 2023, the disclosure of which is hereby incorporated in its entirety by reference herein.

### Technical Field

This case relates to a robot and a monitoring method thereof, and particularly relates to a UV sterilization system and a monitoring method.

### BACKGROUND

### Related Art

A commonly used means for disinfection and sterilization of articles is mainly to use alcohol or disinfectants. The disinfectant is wiped onto the surface of a target object, thereby reducing the number of viruses or bacteria on the surface of the target object. However, for a larger field, cleaning personnel are unable to wipe the surface of every object within the field.

In order to clean and disinfect a large field (hereinafter referred to as sterilization), an ultraviolet (UV) disinfecting device is mostly used. The UV disinfecting device needs to ensure that the irradiation dose of UV exceeds a disinfection threshold to achieve the purpose of disinfection. The irradiation dose of UV depends on the irradiation energy and the irradiation duration of a UV lamp. However, UV can damage the structures of deoxyribonucleic acid (DNA) and ribonucleic acid (RNA) of organisms, including pathogens such as viruses or bacteria. Similarly, UV can cause the same harm to human bodies. Therefore, the UV disinfecting device usually operates in an unmanned environment. In other words, in the working process of the UV disinfecting device, a user is unable to confirm the working state of the UV lamp on site. Therefore, if any UV lamp in the UV disinfecting device is damaged or decayed, the irradiation dose will be reduced and the pathogens cannot be effectively eliminated.

### SUMMARY

In view of this, this case provides a UV sterilization system and a monitoring method, which can automatically identify the working state of a UV lamp, for example whether any UV lamp has been damaged or decayed. In some embodiments, the UV sterilization system or the monitoring method is also suitable for automatically detecting the irradiation intensity of the UV lamp when it begins to decay, and adjusting the travel speed of an autonomous mobile robot accordingly, thereby achieving a predetermined irradiation dose.

In an embodiment, the UV sterilization system is configured to record the working state of the autonomous mobile robot during UV sterilization, and the sterilization system includes a communication module, a UV lamp group, photoinduction elements and a control module. The UV lamp group is configured to emit UV; the photoinduction elements are located on the periphery of the UV lamp group, and are configured to generate a photoinduced voltage value of the corresponding UV lamp group; the control module is connected to the communication module and the photoinduction elements, and is configured to generate a report according to the photoinduced voltage value of the photoinduction element and output the report via the communication module, where the report indicates the working state of the UV lamp group.

In some embodiments, the UV lamp group includes a plurality of UV lamps and a drive circuit, where each UV lamp is configured to emit the UV, the drive circuit is connected to the plurality of UV lamps and the control module, the control module drives the plurality of UV lamps via the drive circuit, and each photoinduction element is located on the periphery of the corresponding UV lamp.

In some embodiments, the report includes an indicator value representing a light source condition of the UV lamp group and record content of the photoinduced voltage value of each UV lamp.

In some embodiments, the indicator value includes a plurality of bits, and the bits correspond to the photoinduced voltage values of the plurality of UV lamps, respectively. The control module determines the corresponding bit in the indicator value based on the magnitude of the photoinduced voltage value of each UV lamp.

In some embodiments, the UV lamp group further includes a plurality of light shields. The light shields correspond to the photoinduction elements respectively, and are each located at one end of the corresponding UV lamp. Each photoinduction element is located at the same end of the corresponding UV lamp and located between the corresponding light shield and the corresponding UV lamp. Each UV lamp is provided with a corresponding light shield.

In some embodiments, the communication module is connected to a monitoring platform via signals, and the monitoring platform receives the report and records state information of the UV lamp group according to the report.

In some embodiments, the aforementioned state information includes the lamp state of each UV lamp, and the aforementioned monitoring platform records the lamp state of the corresponding UV lamp with the photoinduced voltage value less than or equal to a startup voltage value as a damaged lamp according to the report.

In some embodiments, the aforementioned monitoring platform records the lamp state of the corresponding UV lamp with a photoinduced voltage value greater than the startup voltage value and less than a normal voltage value as a decayed lamp according to the report, and generates and sends an adjustment command corresponding to the photoinduced voltage value of the UV lamp to the autonomous mobile robot. At this time, the control module can receive the adjustment command via the communication module and control a moving apparatus to lower the travel speed according to the adjustment command.

In some embodiments, the aforementioned state information includes the usage time of each UV lamp, and the aforementioned monitoring platform accumulates the usage time of the corresponding UV lamp with the photoinduced voltage value not less than the startup voltage value according to the report.

In some embodiments, the state information includes the photoinduced voltage value of each UV lamp in the report received each time, and the monitoring platform records the photoinduced voltage value of each UV lamp also according to the report, and records an average voltage value of all the UV lamps during a time cycle, where the monitoring platform obtains the average voltage value according to the plurality of photoinduced voltage values of all the UV lamps, and the monitoring platform periodically records the average voltage of each time cycle.

In some embodiments, the monitoring method for UV sterilization includes enabling an autonomous mobile robot to move at a travel speed; driving a plurality of UV lamps of the autonomous mobile robot to emit UV; acquiring a photoinduced voltage value of each UV lamp; generating a report according to the photoinduced voltage value for each UV lamp; and transmitting, by the autonomous mobile robot, the report to a monitoring platform via a network.

In some embodiments, the report includes an indicator value, and the indicator value represents a light source condition of the UV lamp group and has a plurality of bits, and the plurality of bits correspond to the plurality of the photoinduced voltage values of the plurality of UV lamps, respectively. The step of generating the report based on the photoinduced voltage value of each UV lamp includes judging whether the photoinduced voltage value of each UV lamp is greater than the startup voltage value or not; recording the bit corresponding to the photoinduced voltage value greater than the startup voltage value in the indicator value as a first value; recording the bit corresponding to the photoinduced voltage value not greater than the startup voltage value in the indicator value as a second value; and recording the indicator value and the photoinduced voltage values of the plurality of UV lamps in the report. The first value is different from the second value.

In some embodiments, the step of acquiring the photoinduced voltage value of each UV lamp includes sensing, in a case that ambient light is shielded, the UV emitted from one end of each UV lamp to correspondingly generate the photoinduced voltage value.

In some embodiments, the monitoring method for UV sterilization may further include receiving an adjustment command from the monitoring platform; and lowering the travel speed of the autonomous mobile robot in response to the adjustment command. The adjustment command is related to at least one photoinduced voltage value of the photoinduced voltage values of the plurality of UV lamps that is less than a normal voltage value.

In some embodiments, the monitoring method for UV sterilization may further include analyzing the report by the monitoring platform to obtain and record state information of each UV lamp.

In some embodiments, the aforementioned state information includes the lamp state of each UV lamp, and the steps of analyzing the report by the monitoring platform to obtain and recording the state information of each UV lamp include judging whether the photoinduced voltage value of each UV lamp is greater than the startup voltage value or not; and recording the lamp state of the corresponding UV lamp with the photoinduced voltage value less than or equal to the startup voltage value as a damaged lamp.

In some embodiments, the step of analyzing the report by the monitoring platform to obtain and record the state information of each UV lamp further includes judging whether the photoinduced voltage value of each UV lamp is less than a normal voltage value or not; recording the lamp state of the UV lamp with the corresponding photoinduced voltage value greater than the startup voltage value and less than the normal voltage value as a decayed lamp; generating an adjustment command according to the photoinduced voltage values of the plurality of UV lamps; transmitting the adjustment command to the autonomous mobile robot by the monitoring platform; and lowering the travel speed of the autonomous mobile robot in response to the adjustment command.

In some embodiments, the monitoring method for UV sterilization may further include generating a notification message to notify that there is the damaged lamp.

In some embodiments, the monitoring method for UV sterilization may further include receiving a startup command from the monitoring platform by the autonomous mobile robot. At this point, the steps of enabling the autonomous mobile robot to move at the travel speed and the step of driving the plurality of UV lamps of the autonomous mobile robot to emit the UV are executed in response to the startup command.

In some embodiments, the monitoring method for UV sterilization may further include receiving a stop command from the monitoring platform by the autonomous mobile robot; and stopping driving the plurality of UV lamps in response to the stop command.

In summary, the UV sterilization system and the monitoring method thereof in any embodiment may be suitable for monitoring the autonomous mobile robot, thereby may achieve unattended disinfection operations. The working state of the UV lamp of the autonomous mobile robot may also be synchronously confirmed when the disinfection operation is performed each time, thereby providing corresponding treatment in a timely manner. In some embodiments, the UV sterilization system or the monitoring method for UV sterilization may also utilizes an indicator value to represent the working state of each UV lamp, thereby quickly identifying whether a UV lamp in the UV lamp group is abnormal or not (such as decayed or damaged). In some embodiments, when any UV lamp of the autonomous mobile robot is abnormal, the UV sterilization system or the monitoring method for UV sterilization may also generate corresponding notification messages and/or correspondingly adjust the travel speed of the autonomous mobile robot, such that the autonomous mobile robot may provide a predetermined irradiation dose. In some embodiments, the UV sterilization system or the monitoring method for UV sterilization may use the light shield to isolate a luminous source (i.e. ambient light) beyond the corresponding UV lamp, thereby avoiding that the photoinduction element is interfered by the light source beyond the corresponding UV lamp.

### BRIEF DESCRIPTION OF THE DRAWINGS

FIG. 1 is an architecture schematic diagram of an UV sterilization system in an embodiment;

FIG. 2 is a functional block diagram of a UV sterilization system in FIG. 1;

FIG. 3 is a functional block diagram of a UV sterilization system of another embodiment in FIG. 1;

FIG. 4 is a flowchart of a monitoring method for UV disinfection in an embodiment;

FIG. 5 is a schematic diagram of an embodiment of disinfection operation of a UV disinfection system;

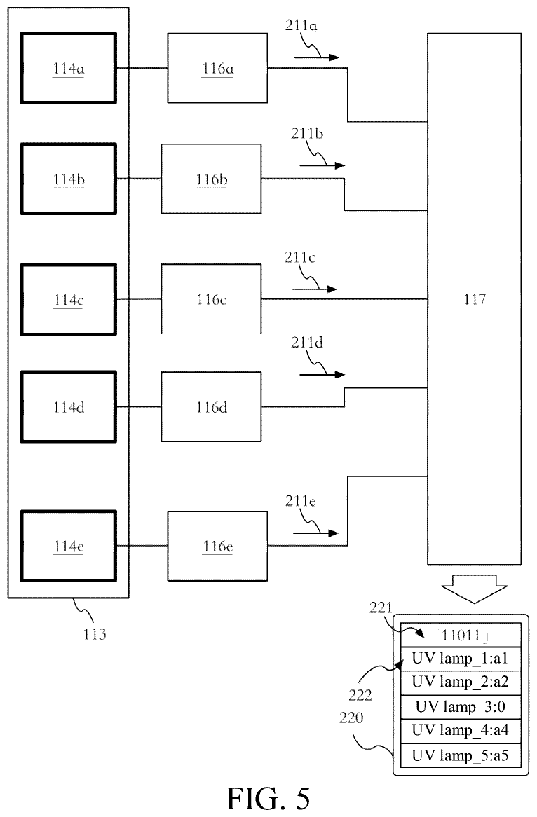

FIG. 6 is a schematic diagram of a corresponding relationship between parameters related to an irradiation dose in an embodiment of a UV disinfection system;

FIG. 7 is a schematic diagram of a corresponding relationship between parameters related to an irradiation dose in another embodiment of a UV disinfection system; and

FIG. 8 is a schematic diagram of an embodiment of an autonomous mobile robot.

### DETAILED DESCRIPTION

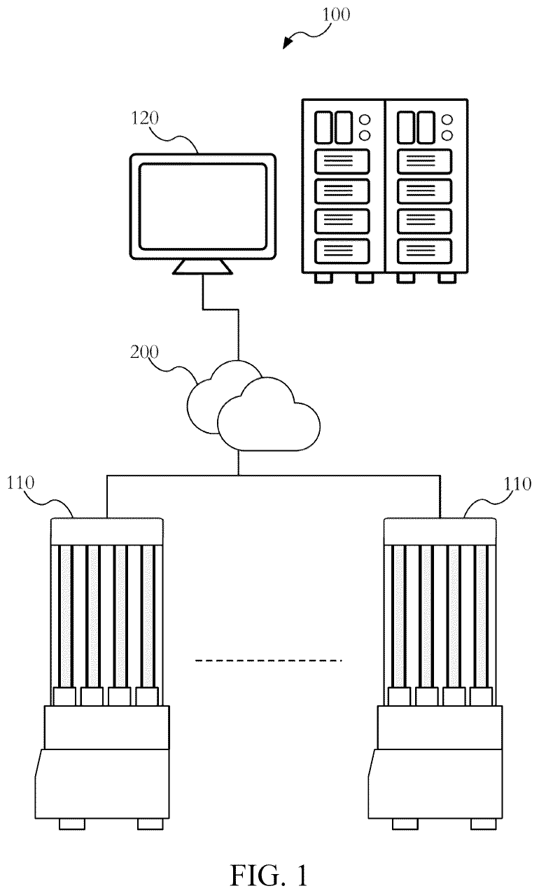

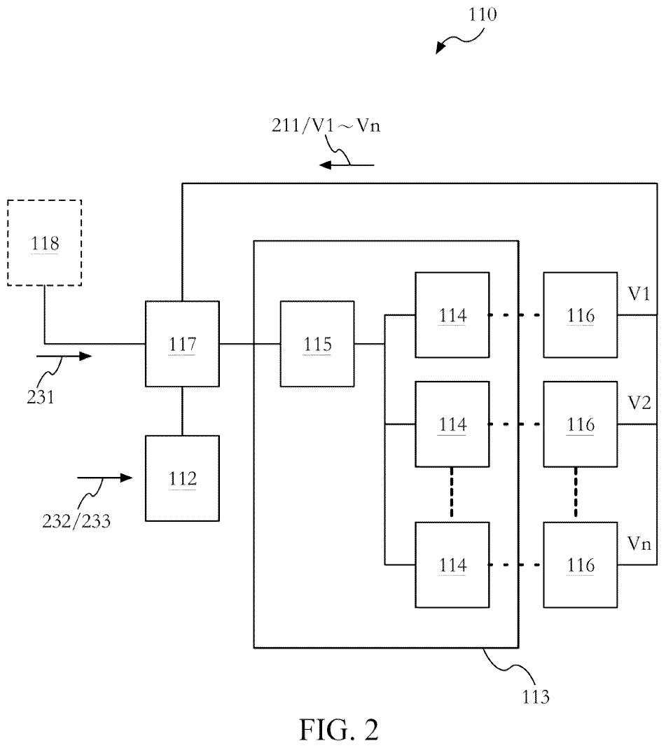

Referring to FIGS. 1 and 2, an ultraviolet (UV) disinfection system 100 is applied to record the working state of an autonomous mobile robot 110 during UV disinfection. The UV sterilization system 100 includes the autonomous mobile robot 110 and a monitoring platform 120. The autonomous mobile robot 110 includes a communication module 112, a UV lamp group 113, photoinduction elements 116 and a control module 117. The control module 117 is connected to the communication module 112, the UV lamp group 113 and the photoinduction elements 116. In some embodiments, the control module 117 may be implemented by one or more processing units. The aforementioned processing unit may be, but is not limited to, a microprocessor, a microcontroller, an embedder controller, a digital signal processor, a central processing unit, a programmable logic controller, or any chip or circuit based on an operating signal of an operating instruction.

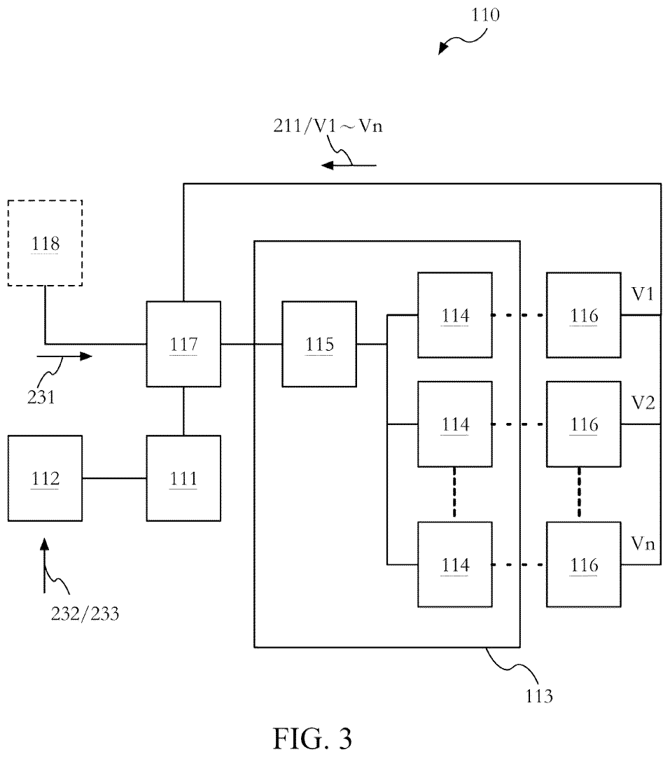

In some embodiments, the autonomous mobile robot 110 further includes a moving apparatus 111, please refer to FIG. 3. The communication module 112, the UV lamp group 113, the photoinduction elements 116 and the control module 117 are located on the moving apparatus 111. Alternatively, the UV lamp group 113 and the photoinduction elements 116 are assembled above the moving apparatus 111. Under the control of the control module 117, the moving apparatus 111 can move at a travel speed or stop moving. Specifically, the moving apparatus 111 includes a moving mechanism, a driver and a housing. The communication module 112 and the control module 117 are provided in the housing of the moving apparatus 111. The driver is coupled between the control module 117 and the moving mechanism. The control module 117 enables the driver of the moving apparatus 111, so that the driver can drive the moving mechanism, such that the moving mechanism moves the entire autonomous mobile robot 110. For example, when the moving apparatus 111 is a vehicle employing moving mechanisms such as tires, crawler belts or mechanical legs, the moving mechanism can be rotatably embedded in the housing of the moving apparatus 111 and located on the side of the housing adjacent to the ground. Alternatively, when the moving apparatus 111 is a flight device such as a drone, the moving mechanism such as a propeller or a turbine is installed above, in front of, or on the side of the housing.

The photoinduction element 116 utilizes a photosensitive element, configured to convert an optical signal into an electrical signal. Alternatively, the photoinduction element 116 may use a photoinduction element such as a complementary metal-oxide-semiconductor (CMOS) or a charge-coupled device (CCD) as a detection element for light ray intensity. The UV lamp group 113 includes a plurality of UV lamps 114 and a drive circuit 115. The drive circuit 115 is connected to the UV lamps 114 and the control module 117. Specifically, the drive circuit 115 is coupled between each UV lamp 114 and the control module 117. The control module 117 enables the drive circuit 115, so that the drive circuit 115 drives the UV lamp 114, causing the UV lamp 114 to emit the UV. In some embodiments, the UV emitted by the UV lamp 114 may be ultra violet C radiation (UV-C).

A photoinduction element 116 is correspondingly arranged on the periphery of each UV lamp 114. In other words, the UV lamps 114 corresponds to the photoinduction elements 116 on a one-to-one basis, and each photoinduction element 116 is located on the periphery of its corresponding UV lamp 114. The number of the UV lamps 114 is the same as that of the photoinduction elements 116. In some embodiments, the UV lamp 114 and the photoinduction element 116 can be assembled on the outer surface of the upper side of the housing of the moving apparatus 111. The drive circuit 115 is assembled on the outer surface of the upper side of the housing or in a position of the housing close to the upper portion.

When the UV lamp 114 is driven to emit UV, each photoinduction element 116 generates a photoinduced voltage value 211 of the corresponding UV lamp 114 by sensing the UV emitted by its corresponding UV lamp 114, and provides the generated photoinduced voltage value 211 to the control module 117. At this point, the photoinduced voltage value 211 can be regarded as the current irradiation intensity of the UV lamp 114, and thereby, the irradiation dose of the current disinfection operation can be then evaluated. Further, the irradiation dose depends on the product of the irradiation intensity and irradiation time of each UV lamp 114 in the current disinfection operation.

Moreover, the control module 117 acquires the photoinduced voltage value 211 of the corresponding UV lamp 114 from the photoinduction element 116. Then, the control module 117 generates a report 220 according to the received photoinduced voltage value 211, and outputs the report 220 via the communication module 112. The report 220 represents the working state of the UV lamp group 113. Specifically, the control module 117 can judge the working state of the corresponding UV lamp 114 (for example whether it has been damaged or decayed) by each photoinduced voltage value 211, and generate a corresponding report 220 accordingly. The communication module 112 may be a wired (or wireless) network element that supports the IEEE802.3 series, Bluetooth, Zigbee or the like.

The autonomous mobile robot 110 can be connected to the monitoring platform 120 via a wireless network. In some embodiments, the autonomous mobile robot 110 and the monitoring platform 120 can communicate (or wirelessly connected) with each other via wireless communication technologies such as Bluetooth, Ultra wideband or ZigBee.

At this point, after the control module 117 generates the report 220, the control module 117 can transmit the report 220 to the monitoring platform 120 via the communication module 112. In this way, the monitoring platform 120 can know and record the state information of the UV lamp group 113 according to the report 220.

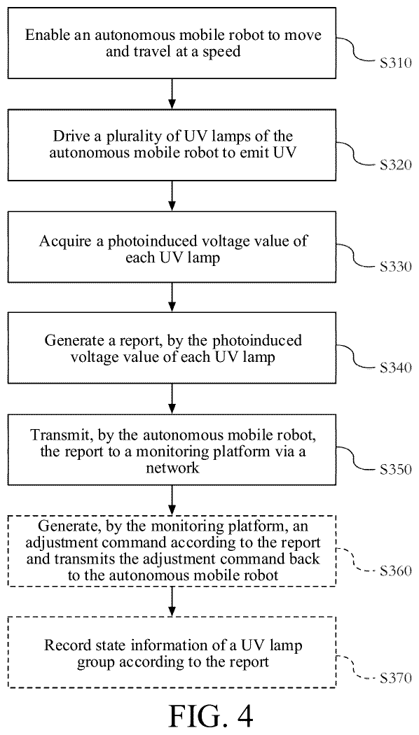

Please refer to FIGS. 1 to 4. In some embodiments, when the UV sterilization system 100 intends to perform a disinfection operation, the control module 117 of the autonomous mobile robot 110 will start up its moving apparatus 111, and thereby, the autonomous mobile robot 110 is enabled to move at a travel speed (step S310). Moreover, the control module 117 of the autonomous mobile robot 110 also starts up the drive circuit 115 to drive the plurality of UV lamps 114 of the autonomous mobile robot 110, so that each UV lamp 114 emits the UV (step S320). At this point, during the operation of the autonomous mobile robot 110, the drive circuit 115 will continuously work, so that the autonomous mobile robot 110 will continuously emit the UV in the movement process, thereby disinfecting the surrounding environment on a travel path.

After the UV lamp 114 is driven (step S320), each photoinduction element 116 will sense the UV emitted by the corresponding UV lamp 114, converts the sensed UV into the photoinduced voltage value 211 corresponding to its irradiation intensity, and then outputs the photoinduced voltage value to the control module 117, such that the control module 117 acquires the photoinduced voltage value 211 of each UV lamp 114 (step S330).

After step S330, the control module 117 generates the report 220 corresponding to the working state of the UV lamp 114 according to the photoinduced voltage value 211 of each UV lamp 114 (step S340). Then, the autonomous mobile robot 110 transmits the report 220 to the monitoring platform 120 via a network 200 (step S350).

In some embodiments, after the monitoring platform 120 receives the report 220 transmitted in step S350, the monitoring platform 120 can generate an adjustment command 232 according to the received report 220 and transmit the adjustment command 232 back to the autonomous mobile robot 110 (step S360).

In some embodiments, steps S310 to S320 can be executed in response to a startup command 231. The startup command 231 may be sent by the monitoring platform 120 to the autonomous mobile robot 110, or can be generated by a schedule set by the autonomous mobile robot 110, or generated correspondingly by a user operating a user interface 118. In an exemplary example, the control module 117 of the autonomous mobile robot 110 may communicate with the monitoring platform 120 via the communication module 112 and receive the startup command 231 from the monitoring platform 120. Next, the control module 117 starts up operations of the moving apparatus 111 and the UV lamp group 113 in response to the startup command 231, such that the autonomous mobile robot 110 starts to move while emitting the UV. In another exemplary example, the control module 117 of the autonomous mobile robot 110 can receive the startup command 231 from the user interface 118 and start up the operations of the moving apparatus 111 and the UV lamp group 113 in response to the startup command 231, such that the autonomous mobile robot 110 starts to move while emitting the UV. The user interface 118 may be implemented by a plurality of physical keys, a touch panel, or a combination of the touch panel and at least one physical key. In yet another exemplary example, when reaching the predetermined execution time of the disinfection operation, the control module 117 of the autonomous mobile robot 110 will execute this schedule to generate the startup command 231, and starts up the operations of the moving apparatus 111 and the UV lamp group 113 in response to the startup command 231, such that the autonomous mobile robot 110 starts to move while emitting the UV.

In some embodiments, the report 220 includes an indicator value 221 representing a light source condition of the UV lamp group 113. The indicator value 221 includes a plurality of bits, and the bits correspond to the photoinduced voltage values 211 of the UV lamps 114, respectively. In step S340, the control module 117 determines, based on the photoinduced voltage value 211 of each UV lamp 114, the corresponding bit in the indicator value 221. In some embodiments, the indicator value 221 may be a binary bit combination. In other words, this bit combination is composed of a plurality of bits, and a content value of each bit is a binary value, that is, 0 or 1. In other embodiments, the indicator value 221 may be a decimal value further converted from the binary bit combination. In other words, the indicator value 221 is a bit combination consisting of a plurality of bits that is represented in decimal.

In some embodiments of step S340, the control module 117 compares the photoinduced voltage value 211 of each UV lamp 114 with the startup voltage value. If the photoinduced voltage value 211 of the UV lamp 114 is greater than the startup voltage value, the control module 117 records the bit corresponding to the UV lamp 114 as a first value, that is, the content value of this bit is a first value. In other words, in step S330, the control module 117 sequentially reads the photoinduced voltage values 211 generated by the photoinduction elements 116, that is, the UV lamp 114 has an order bit individually due to the order in which the photoinduced voltage values 211 are acquired. At this time, in step S340, the control module 117 after comparison, judges that the photoinduced voltage value 211 of the UV lamp 114 is greater than the startup voltage value, then the first value is written into a corresponding position in the indicator value 221 according to the order bit in which the photoinduced voltage value 211 of the UV lamp 114 is acquired (that is, the order of the corresponding bit in the bit combination). On the contrary, if the photoinduced voltage value 211 of the UV lamp 114 is less than or equal to the startup voltage value, the control module 117 records the bit corresponding to the UV lamp 114 as a second value. In other words, the control module 117 writes the second value into a corresponding position in the indicator value 221 according to the order bit in which the photoinduced voltage value 211 of the UV lamp 114 is acquired (that is, the order of the corresponding bit in the bit combination).

In some embodiments, the control module 117 also records the photoinduced voltage value 211 of each UV lamp 114 in the report 220. In other words, the report 220 further includes the record content of the photoinduced voltage value 211 of each UV lamp 114.

In an embodiment, the startup voltage value may be the photoinduced voltage value 211 when the UV lamp 114 does not emit UV. In another embodiment, the startup voltage value is equivalent to the lower limit illuminance of the UV lamp 114 at the irradiation intensity at which disinfection can be performed. In other words, the startup voltage value may be the photoinduced voltage value 211 of the UV lamp 114 that emits a UV but the irradiation intensity of which cannot provide an expected irradiation dose. Under normal circumstances, as long as the operable voltage is provided to the UV lamp 114, the UV lamp 114 can be enabled to emit UV. However, in a case that the photoinduced voltage value 211 is less than the startup voltage value, the irradiation dose provided by the irradiation intensity of the UV lamp 114 still does not meet the minimum requirement for disinfection, that is, the product of the irradiation intensity and specified time is less than the expected irradiation dose. Specifically, after the UV lamp 114 with the photoinduced voltage value 211 less than the startup voltage value continuously irradiates for a specified time, the irradiation dose applied by the UV lamp to an irradiated target still cannot reach the predetermined irradiation dose. At this point, the autonomous mobile robot 110 can preset the corresponding startup voltage value according to the model of the UV lamp 114 used by the autonomous mobile robot for the control module 117 to confirm the working state of each UV lamp 114.

By way of examples, when the UV lamp 114 has been damaged and cannot be enabled (i.e. powered up but still not emitting UV), the photoinduced voltage value 211 of the UV lamp 114 is “0 (mW/cm²)”. The autonomous mobile robot 110 can set the startup voltage value as “0 (mW/cm²)” accordingly. Therefore, after the UV lamp group 113 is enabled, if the photoinduced voltage value 211 of any UV lamp 114 is not greater than the startup voltage value, the control module 117 will set the bit corresponding to this UV lamp 114 in the indicator value 221 as “0” (i.e., the second value). On the contrary, after the UV lamp group 113 is enabled, if the photoinduced voltage value 211 of the UV lamp 114 is greater than the startup voltage value, it indicates that the UV lamp can be enabled and emit UV normally. At this time, the control module 117 will set the bit corresponding to the UV lamp 114 in the indicator value 221 as “1” (i.e., the first value).

By way of examples, referring to FIGS. 2 (or FIG. 3) and 5, it is assumed that the UV lamp group 113 has 5 UV lamps 114a to 114e, and 5 photoinduction elements 116a to 116e are provided on a one-to-one basis on the periphery of the UV lamps 114a to 114e. Moreover, the photoinduced voltage value 211 when the UV lamp 114 does not emit UV is “0 (mW/cm²)”, and therefore, the startup voltage value is preset as “0”.

In an exemplary example, after the UV lamp group 113 is enabled, the UV lamps 114a, 114b, 114d and 114e emit UV, while the UV lamp 114c does not emit UV. At this time, the photoinduced voltage value 211a outputted by the photoinduction element 116a is “a1”, the photoinduced voltage value 211b outputted by the photoinduction element 116b is “a2”, the photoinduced voltage value 211c outputted by the photoinduction element 116c is “0”, the photoinduced voltage value 211d outputted by the photoinduction element 116d is “a4”, and the photoinduced voltage value 211e outputted by the photoinduction element 116e is “a5”. Moreover, a1, a2, a4 and a5 are not 0. Moreover, in step S340, the control module 117 will compare the photoinduced voltage values 211a to 211e individually with “0” (i.e. the startup voltage value) to obtain that the photoinduced voltage values 211a, 211b, 211d and 211e are greater than the startup voltage value and the photoinduced voltage value 211c is equal to the startup voltage value, thus generating a bit combination of “11011”.

When the indicator value 221 is represented in binary, the control module 117 directly uses the generated bit combination as the indicator value 221, and adds the indicator value 221 of “11011” and the record content 222 in which the photoinduced voltage values 211a to 211e are “a1”, “a2”, 0, “a4” and “a5”, respectively, into the report 220. When the indicator value 221 is represented in decimal, the control module 117 then converts the generated bit combination into a decimal value of “27” as the indicator value 221, and adds the indicator value 221 of “27” and the record content 222 in which the photoinduced voltage values 211a to 211e are “a1”, “a2”, 0, “a4” and “a5”, respectively, into the report 220.

In another exemplary example, after the UV lamp group 113 is enabled, its UV lamps 114a to 114e do not emit UV, then the photoinduced voltage values 211a to 211e of the UV lamps 114a to 114e acquired by the control module 117c are all “0”, thus generating a bit combination of “00000” (meaning that a decimal value of “0” is generated).

In yet another exemplary example, after the UV lamp group 113 is enabled, its UV lamps 114a to 114e all emit UV, then the photoinduced voltage values 211a to 211e of the UV lamps 114a to 114e acquired by the control module 117c are all values greater than “0”, thus generating a bit combination of “11111” (or a decimal value of “31” is generated).

In some embodiments, after the monitoring platform 120 receives the report 220 transmitted in step S350, the monitoring platform 120 also records the state information of the UV lamp group 113 according to the report 220 (step S370). In other words, the monitoring platform 120 stores the state information of the autonomous mobile robot 110. After receiving the report 220, the monitoring platform 120 analyzes the record content related to the state of the UV lamp group 113 in the report 220 to judge the working state that the UV lamp group 113 can provide and adds the judgment result to the state information of the corresponding autonomous mobile robot 110.

In some embodiments, the state information of the UV lamp group 113 includes the lamp state of each UV lamp 114. Therefore, in step S370, the monitoring platform 120 will analyze the report 220 to obtain and record the lamp state of each UV lamp 114 accordingly.

In some exemplary patterns of step S370, the monitoring platform 120 acquires the photoinduced voltage value 211 of each UV lamp 114 from the report 220, and judges whether the photoinduced voltage value 211 conforms to the startup voltage value or not.

When the photoinduced voltage value 211 of the UV lamp 114 is less than or equal to the startup voltage value, the monitoring platform 120 obtains and records the lamp state of the UV lamp 114 as a damaged lamp (unlabeled). In an embodiment, the startup voltage value may be the photoinduced voltage value 211 when the UV lamp 114 does not emit UV. In another embodiment, the startup voltage value may be the photoinduced voltage value 211 of the UV lamp 114 that emits UV but the irradiation intensity of which cannot provide the expected irradiation dose. In other words, under normal circumstances, as long as the operable voltage is provided to the UV lamp 114, the UV lamp 114 can be enabled to emit UV. However, in a case that the photoinduced voltage value 211 is less than the startup voltage value, the irradiation dose provided by the irradiation intensity of the UV lamp 114 still does not meet the minimum requirement for disinfection, that is, the product of the irradiation intensity and specified time is less than the expected irradiation dose. In other words, the UV lamp 114 with the photoinduced voltage value 211 less than the startup voltage value cannot reach the predetermined irradiation dose after continuously irradiating for a specified time. At this point, the monitoring platform 120 sets the corresponding startup voltage value according to the model of the UV lamp 114 used by the autonomous mobile robot 110 for confirming the lamp state of each UV lamp 114.

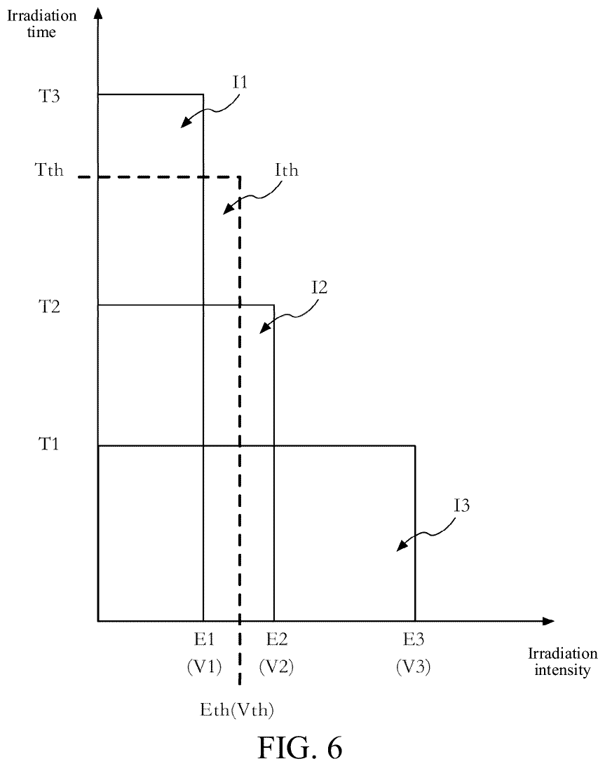

By way of examples, referring to FIGS. 1 and 6, three UV lamps 114 are taken as an example. The photoinduced voltage values 211 of the three UV lamps 114 correspond to irradiation intensities E1, E2, and E3, respectively. The irradiation intensity E3 is greater than the irradiation intensity E2, and the irradiation intensity E2 is greater than the irradiation intensity E1. An irradiation dose Ith (i.e., the area enclosed by irradiation intensity Eth and time Tth, Ith=Eth*Tth) is a predetermined target amount. Therefore, the irradiation intensity E3 requires an irradiation dose I3 provided by the irradiation time T1 (i.e. the area enclosed by the irradiation intensity E3 and the time T1, I3=E3*T1) to be equivalent to the irradiation dose Ith. The irradiation intensity E2 requires an irradiation dose I2 provided by irradiation time T2 (i.e. the area enclosed by the irradiation intensity E2 and the time T2, I2=E2*T2) to be equivalent to the irradiation dose Ith. The irradiation intensity E1 requires an irradiation dose I1 provided by irradiation time T3 (i.e. the area enclosed by the irradiation intensity E1 and the time T3, I1=E1*T3) to be equivalent to the irradiation dose Ith. The time T1 is less than the time T2, and the time T2 is less than the time T3.

In other words, in order to reach the predetermined irradiation dose Ith, the irradiation time T3 of the UV lamp 114 with the irradiation intensity E1 will exceed the specified time Tth, indicating that the irradiation dose I1 provided by the UV lamp 114 with the irradiation intensity E1 cannot reach the predetermined irradiation dose Ith within the specified time Tth. Therefore, a photoinduced voltage value Vth of the UV lamp 114 with the irradiation intensity Eth is set as a startup voltage value (Vth) in advance. That is, by comparing the photoinduced voltage values V1/V2/V3 of all the UV lamps 114 with the startup voltage value (Vth), it can be determined that which UV lamp 114 has been damaged, and then its lamp state is recorded as the damaged lamp. Specifically, the photoinduced voltage value V1 of the UV lamp 114 with the irradiation intensity E1 is less than the startup voltage value (Vth), then the monitoring platform 120 judges that the UV lamp 114 with the irradiation intensity E1 has been damaged and its lamp state is recorded as the damaged lamp. The photoinduced voltage values V2 and V3 of the UV lamps 114 with the irradiation intensities E2 and E3 are greater than the startup voltage value (Vth), then the monitoring platform 120 judges that the UV lamps 114 with the irradiation intensities E2 and E3 are undamaged.

In some exemplary patterns of step S370, in addition to recording the lamp state of the damaged UV lamp 114, the monitoring platform 120 will also judge that the UV lamp 114 with the photoinduced voltage value 211 greater than the startup voltage value (Vth) is undamaged and record the lamp state of the UV lamp 114 that has been determined to be undamaged as a working lamp.

In some embodiments, after the monitoring platform 120 judges that there has been a damaged UV lamp 114 in the UV lamp group 113 of the autonomous mobile robot 110, the monitoring platform 120 will generate a notification message to notify that there is a damaged lamp. Next, the monitoring platform 120 sends the generated notification message to a designated client, and thereby, a message that there is a damaged UV lamp 114 in the autonomous mobile robot 110 is generated on the client to notify the user.

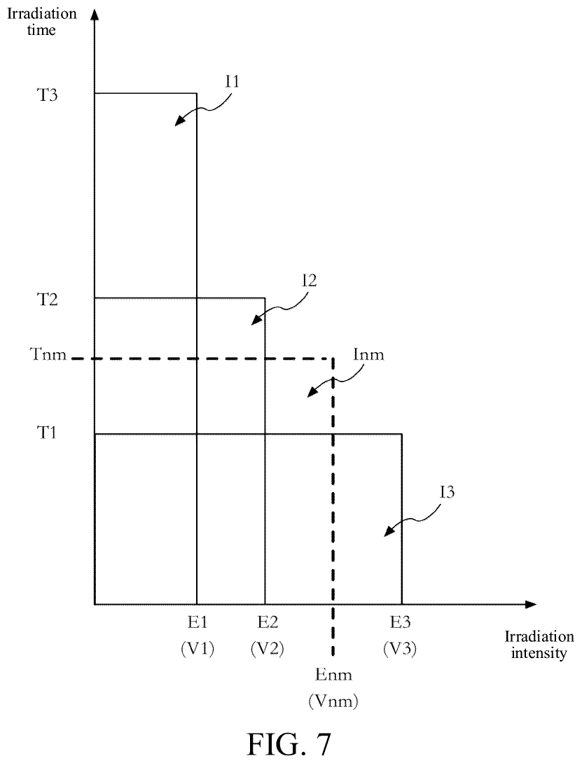

In some exemplary patterns of step S370, in addition to judging by the monitoring platform 120 whether the startup voltage value is conformed or not to find out the damaged UV lamp 114 (that is, its lamp state is the damaged lamp), the monitoring platform 120 judges whether the normal voltage value is conformed or not to find out the decayed UV lamp 114 (that is, its lamp state is the decayed lamp). After the UV lamp 114 is put into use, its irradiation intensity will decay with usage time. Referring to FIGS. 1 and 7, when the irradiation intensity is greater than or equal to a critical value, the UV lamp 114 can reach the target amount of irradiation dose within an ideal time, i.e., Ei≥Enm, Ti≤Tnm, and Ii≥Inm. i is a positive integer. Similarly, when the irradiation intensity is less than this critical value, the UV lamp 114 will unable to reach the target amount of irradiation dose within the ideal time, i.e., Ei<Enm, Ti≤Tnm, and Ii<Inm. The photoinduced voltage value 211 of the UV lamp 114 with the irradiation intensity Enm of the critical value can be set as a normal voltage value Vnm. The normal voltage value Vnm is greater than the startup voltage value Vth.

Specifically, after acquiring the photoinduced voltage value 211 of each UV lamp 114 from the report 220, the monitoring platform 120 will compare the photoinduced voltage value 211 of each UV lamp 114 with the startup voltage value Vth and the normal voltage value Vnm. Then, the monitoring platform 120 judges that the UV lamp 114 with the photoinduced voltage value 211 less than or equal to the startup voltage value Vth has damaged and records the lamp state of the UV lamp 114 as the damaged lamp. Moreover, the monitoring platform 120 also judges that the UV lamp 114 with the photoinduced voltage value 211 greater than the startup voltage value Vth and less than the normal voltage value Vnm has been decayed, and records the lamp state of the UV lamp 114 as the decayed lamp. Based on this, the monitoring platform 120 can learn and record an abnormal UV lamp 114 of the UV lamps 114 of the autonomous mobile robot 110 and the type of the abnormality thereof by the report 220, and then queries are provided to the user to perform corresponding repairs on the autonomous mobile robot 110 to eliminate the abnormality.

In some exemplary patterns of step S370, in addition to correspondingly recording the lamp states of the abnormal (i.e. damaged or decayed) UV lamps 114, the monitoring platform 120 will also judge that the UV lamp 114 with the photoinduced voltage value 211 greater than or equal to the normal voltage value Vnm is normal and records the lamp state of the UV lamp 114 that has been determined to be normal as a normal lamp.

In some exemplary patterns of step S370, after the monitoring platform 120 judges that there is a decayed UV lamp 114 in the UV lamp group 113 of the autonomous mobile robot 110, the monitoring platform 120 will generate and send an adjustment command 232 corresponding to the photoinduced voltage value 211 of the UV lamp 114 to the autonomous mobile robot 110. At this time, the control module 117 of the autonomous mobile robot 110 can receive the adjustment command 232 from the monitoring platform 120 via the communication module 112, and control the moving apparatus 111 to lower the travel speed according to the adjustment command 232.

In some embodiments, the monitoring platform 120 can convert the total irradiation intensity currently provided by the UV lamp group 113 according to the photoinduced voltage value 211 of the decayed UV lamp 114 and the photoinduced voltage value 211 of the normal UV lamp 114, and calculate required minimum irradiation time by the total irradiation intensity and the predetermined irradiation dose (i.e., target amount). Next, the monitoring platform 120 then generates a new travel speed according to the minimum irradiation time acquired, and generates an adjustment command 232 at the new travel speed.

Therefore, the control module 117 of the autonomous mobile robot 110 can update the travel speed of the moving apparatus 111 to the new travel speed indicated by the adjustment command 232 according to the adjustment command 232, such that the moving apparatus 111 moves at the updated travel speed. In some embodiments, the monitoring platform 120 may be preset with a unit distance corresponding to the autonomous mobile robot 110. In the process of the autonomous mobile robot 110 moving for the unit distance, the UV emitted by autonomous mobile robot 110 can continuously irradiate upon the same target point. Therefore, the monitoring platform 120 can calculate a threshold value of travel speed by the minimum irradiation time and the unit distance, and generate a new travel speed based on the allowable range and threshold value of the travel speed of the autonomous mobile robot 110. That is, the new travel speed is less than or equal to the threshold value and falls within the allowable range.

In some exemplary patterns of step S370, after the monitoring platform 120 judges that there is a decayed UV lamp 114 in the UV lamp group 113 of the autonomous mobile robot 110, the monitoring platform 120 will also generate a notification message to notify that there is a decayed lamp. Next, the monitoring platform 120 will send the generated notification message to a designated client, and thereby, a message that there is a decayed UV lamp 114 in the autonomous mobile robot 110 is generated on the client to notify the user.

In some embodiments, the state information of the UV lamp group 113 may further include the usage time of each UV lamp 114. After the monitoring platform 120 receives the report 220 transmitted in step S350, the monitoring platform 120 can also accumulate the usage time for each UV lamp 114 according to the report 220.

In some embodiments, the monitoring platform 120 specially accumulates the usage time of the UV lamp 114 with the photoinduced voltage value 211 not less than the startup voltage value Vth. By way of examples, the monitoring platform 120 calculates a time difference from the time of acquiring the report 220 this time and the time of acquiring the report 220 previously, and then calculates the sum of the usage time of the undamaged UV lamps 114 in the stored state information of the UV lamp group 113 and time difference to obtain the update time of the undamaged UV lamp 114, and updates the usage time of the corresponding UV lamp 114 in its stored state information of the UV lamp group 113 with the update time of the undamaged UV lamp 114.

In some embodiments, the state information of the UV lamp group 113 may further include the photoinduced voltage value 211 of each UV lamp 114 in the report 220 received each time. In other words, after the monitoring platform 120 receives the report 220 transmitted in step S350, the monitoring platform 120 can acquire the photoinduced voltage value 211 of each UV lamp 114 from the report 220, and record the photoinduced voltage value 211 of each UV lamp 114 in the stored state information of the UV lamp group 113 corresponding to the time of receiving the report 220.

In some embodiments, the state information of the UV lamp group 113 may further include an average voltage value of all the photoinduced voltage values 211 recorded for all the UV lamp 114 in each time cycle. In other words, the monitoring platform 120 periodically receives the report 220 from the autonomous mobile robot 110 (i.e., the report 220 transmitted in step S350) and records the photoinduced voltage value 211 of each UV lamp 114 therein in the state information of the UV lamp group 113. Then, after each time cycle, the monitoring platform 120 will also calculate and record the average voltage value of all the photoinduced voltage values 211 of all the UV lamps 114 recorded in this time cycle. Each elapsed time cycle may be a same duration or a random duration. In other words, the monitoring platform 120 periodically calculates the average voltage of each UV lamp 114 recorded in a time cycle. In some embodiments, the monitoring platform 120 can provide the average voltage values of each UV lamp 114 recorded at different times to the client for the user to judge whether the UV lamp group 113 is decayed or not. When the average voltage value is abnormal, it can be considered that any UV lamp 114 in the UV lamp group 113 has decayed or cannot emit the UV.

In some embodiments, the time cycle may, based on days, weeks, months, seasons, or years, serve as a timing duration of the average voltage value of each UV lamp 114.

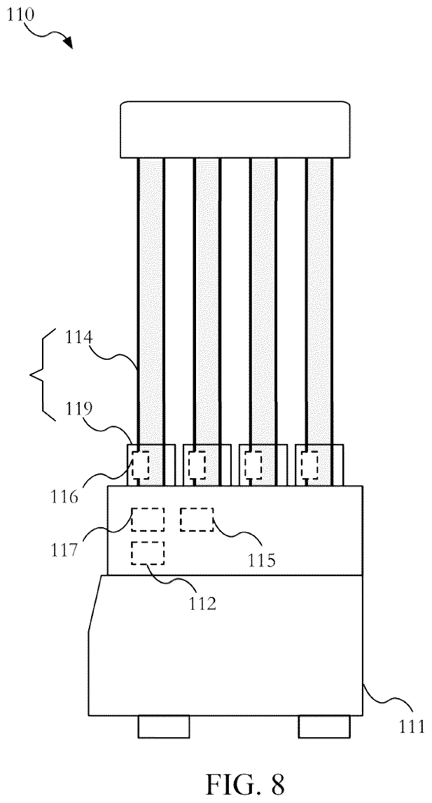

In some embodiments, referring to FIGS. 1 and 8, the UV lamp group 113 may further include a plurality of light shields 119. The light shields 119 correspond to the UV lamps 114 on a one-to-one basis, that is, the number of the light shields 119 is equal to that of the UV lamps 114. Each light shield 119 is provided at one end of the corresponding UV lamp 114, and the photoinduction element 116 is located between the corresponding light shield 119 and the corresponding UV lamp 114. At this point, each light shield 119 is located in the periphery position of one end of the corresponding UV lamp 114. The edge of the light shield is attached to the UV lamp 114, and the middle section of its shield body is at a fixed distance from the corresponding UV lamp 114, such that a holding space is sandwiched between each light shield 119 and the corresponding UV lamp 114. The corresponding photoinduction element 116 is provided in the holding space. At this point, the light shield 119 is configured to shield the ambient light (such as the UV of adjacent UV lamps 114) to avoid that the ambient light interferes with the corresponding photoinduction element 116. For example, the light shield 119 can avoid that the UV projected by the adjacent UV lamps 114 influences the detection accuracy of the corresponding photoinduction element 116 to generate the photoinduced voltage value 211 of the corresponding UV lamp 114. In other words, each photoinduction element 116 senses the UV emitted from one end of the corresponding UV lamp 114 in a case that the ambient light is shielded, whereby the photoinduced voltage value 211 of this UV lamp 114 is correspondingly generated.

In some embodiments, the stop time of the autonomous mobile robot 110 can be controlled by the monitoring platform 120. Specifically, when the disinfection operation of the autonomous mobile robot 110 is intended to be stopped, the monitoring platform 120 can generate and send a stop command 233 to the autonomous mobile robot 110. After the autonomous mobile robot 110 receives the stop command 233 from the monitoring platform 120, the autonomous mobile robot 110 will stop driving the UV lamp group 113 to emit the UV (i.e., disabling the drive circuit 115 of the UV lamp group 113) or/and stop the operation of the moving apparatus 111 (i.e., disabling the driver of the moving apparatus 111) in response to the stop command 233. At the same time, the autonomous mobile robot 110 encapsulates the photoinduced voltage values 211 of the UV lamp group 113 as the report 220. Since the UV lamp group 113 does not emit UV, its bit combination is “00000”. The autonomous mobile robot 110 sends the report 220 to the monitoring platform 120 in order to make the monitoring platform 120 stop counting the usage time of each UV lamp 114.

In summary, the UV sterilization system 100 and the monitoring method thereof in any embodiment are suitable for monitoring the autonomous mobile robot 110, thereby achieving unattended disinfection operations. The working state of the UV lamp 114 of the autonomous mobile robot 110 can also be synchronously confirmed when the disinfection operation is performed each time, thereby providing corresponding treatment in a timely manner to ensure the implementation effect of the disinfection operations. In some embodiments, the UV sterilization system 100 or the monitoring method for UV sterilization also utilizes an indicator value 221 to represent the working state of each UV lamp 114, thereby quickly identifying whether a UV lamp 114 in the UV lamp group 113 is abnormal or not (such as decayed or damaged). In some embodiments, when any UV lamp 114 of the autonomous mobile robot 110 is abnormal, the UV sterilization system 100 or the monitoring method for UV sterilization can also generate corresponding notification messages and/or correspondingly adjust the travel speed of the autonomous mobile robot 110, thereby ensuring that the autonomous mobile robot 110 can provide a predetermined irradiation dose. Thus it is ensured that the disinfection operation can effectively eliminate pathogens. In some embodiments, the UV sterilization system 100 or the monitoring method for UV sterilization uses the light shield 119 to isolate a luminous source (i.e. ambient light) beyond the corresponding UV lamp 114, thereby avoiding that the photoinduction element 116 is interfered by the light source beyond the corresponding UV lamp 114, and thus, misjudging the working state of the UV lamp 114 is then avoided.

What is claimed is:

1. An ultraviolet (UV) sterilization system, for recording a working state of an autonomous mobile robot during UV sterilization, the sterilization system comprising:
a communication module;
an UV lamp group, configured to emit UV light;
a photoinduction element, located on a periphery of the UV lamp group, and generating a photoinduced voltage value of the corresponding UV lamp group; and
a control module, provided on the autonomous mobile robot, the control module being connected to the communication module and the photoinduction element, and generating a report according to the photoinduced voltage value of the photoinduction element and outputting the report via the communication module, wherein the report represents the working state of the UV lamp group.

2. The UV sterilization system according to claim 1, wherein the UV lamp group comprises a plurality of UV lamps and a drive circuit, each of the plurality of UV lamps is configured to emit the UV light, the drive circuit is connected to the plurality of UV lamps and the control module, the plurality of UV lamps are driven by the control module via the drive circuit, and the photoinduction element is located around one corresponding UV lamp of the plurality of UV lamps.

3. The UV sterilization system according to claim 2, wherein the report comprises an indicator value representing a light source condition of the UV lamp group and record content of the photoinduced voltage value of each of the plurality of UV lamps.

4. The UV sterilization system according to claim 3, wherein the indicator value comprises a plurality of bits, corresponding to the photoinduced voltage values of the plurality of UV lamps respectively, wherein the corresponding bit in the indicator value based on a magnitude of each of the photoinduced voltage values to each of the plurality of UV lamps is determined by the control module.

5. The UV sterilization system according to claim 2, wherein the UV lamp group further comprises:
a plurality of light shields, each of the plurality of light shields being corresponding to the photoinduction element respectively, and each of the plurality of light shields being located at one end of one corresponding UV lamp of the plurality of UV lamps, wherein the photoinduction element is located at the end of one corresponding UV lamp of the plurality of UV lamps, and located between one corresponding light shield of the plurality of light shields and one corresponding UV lamp of the plurality of UV lamps.

6. The UV sterilization system according to claim 2, wherein the communication module is in signal communication with a monitoring platform, the monitoring platform receives the report is received by the monitoring platform, and state information of the UV lamp group according to the report is recorded by the monitoring platform.

7. The UV sterilization system according to claim 6, wherein the state information comprises a lamp state of each of the plurality of UV lamp, the lamp state of one corresponding UV lamp of the plurality of UV lamps is recorded by the monitoring platform, and one corresponding UV lamp the plurality of UV lamps with the photoinduced voltage value less than or equal to a startup voltage value is recorded by the monitoring platform as a damaged lamp according to the report.

8. The UV sterilization system according to claim 7, wherein the lamp state of one corresponding UV lamp of the plurality of UV lamps with the photoinduced voltage value greater than the startup voltage value and less than a normal voltage value is recorded by the monitoring platform as a decayed lamp according to the report, and an adjustment command corresponding to the photoinduced voltage values of the plurality of UV lamps is generated and sent by the monitoring platform to the autonomous mobile robot, and the adjustment command is received by the control module via the communication module, and the autonomous mobile robot is controlled to lower a travel speed according to the adjustment command by the monitoring platform.

9. The UV sterilization system according to claim 7, wherein the state information further comprises a usage time of each of the plurality of UV lamp, and the usage time of one corresponding UV lamp of the plurality of UV lamps with the photoinduced voltage value not less than the startup voltage value according to the report is accumulated by the monitoring platform.

10. The UV sterilization system according to claim 6, wherein the state information further comprises the photoinduced voltage value of each of the plurality of UV lamp in the report received each time, and the photoinduced voltage value of each of the plurality of UV lamp is recorded by the monitoring platform also according to the report, and an average voltage value of all the plurality of UV lamps is recorded by the monitoring platform during a time cycle, wherein the average voltage value is obtained by the monitoring platform according to the photoinduced voltage values of all the plurality of UV lamps, and the average voltage of each time cycle is periodically recorded by the monitoring platform.

11. A monitoring method for ultraviolet (UV) sterilization, comprising:
enabling an autonomous mobile robot to move at a travel speed;
driving a plurality of UV lamps of the autonomous mobile robot to emit UV light;
acquiring a photoinduced voltage value of each of the plurality of UV lamps;
generating a report, by the photoinduced voltage value of each of the plurality of UV lamps; and
transmitting, by the autonomous mobile robot, the report to a monitoring platform via a network.

12. The monitoring method for UV sterilization according to claim 11, wherein the report comprises an indicator value representing a light source condition of the plurality of UV lamps and including a plurality of bits, the plurality of bits correspond to the photoinduced voltage values of the plurality of UV lamps, respectively, and generating the report based on the photoinduced voltage value of each of the plurality of UV lamps comprises:
judging whether the photoinduced voltage value of each of the plurality of UV lamps is greater than a startup voltage value;
recording the bit corresponding to the photoinduced voltage value greater than the startup voltage value in the indicator value as a first value;
recording the bit corresponding to the photoinduced voltage value not greater than the startup voltage value in the indicator value as a second value, wherein the first value is different from the second value; and
recording the indicator value and the photoinduced voltage values of the plurality of UV lamps in the report.

13. The monitoring method for UV sterilization according to claim 11, wherein acquiring the photoinduced voltage value of each of the plurality of UV lamps comprises:
sensing, in a case that ambient light is shielded, the UV light emitted from one end of each of the plurality of UV lamps to correspondingly generate the photoinduced voltage value.

14. The monitoring method for UV sterilization according to claim 11, further comprising:
receiving an adjustment command from the monitoring platform, wherein the adjustment command is related to at least one photoinduced voltage value of the photoinduced voltage values of the plurality of UV lamps that is less than a normal voltage value; and
lowering the travel speed of the autonomous mobile robot in response to the adjustment command.

15. The monitoring method for UV sterilization according to claim 11, further comprising:
obtaining and recording state information of each of the plurality of UV lamps by analyzing the report via the monitoring platform.

16. The monitoring method for UV sterilization according to claim 15, wherein the state information comprises a lamp state of each of the plurality of UV lamps, and obtaining and recording the state information of each of the plurality of UV lamps by analyzing the report via the monitoring platform comprises:
judging whether the photoinduced voltage value of each of the plurality of UV lamps is greater than a startup voltage value; and
recording the lamp state of one corresponding UV lamp of the plurality of UV lamps with the photoinduced voltage value less than or equal to the startup voltage value as a damaged lamp.

17. The monitoring method for UV sterilization according to claim 16, further comprising:
notifying the damaged lamp by generating a notification message.

18. The monitoring method for UV sterilization according to claim 16, wherein obtaining and recording the state information of each of the plurality of UV lamps by analyzing the report via the monitoring platform further comprises:
judging whether the photoinduced voltage value of each of the plurality of UV lamps is less than a normal voltage value;
recording the lamp state of one corresponding UV lamp of the plurality of UV lamps with the photoinduced voltage value greater than the startup voltage value and less than the normal voltage value as a decayed lamp;
generating an adjustment command according to the photoinduced voltage values of the plurality of UV lamps;
transmitting, by the monitoring platform, the adjustment command to the autonomous mobile robot; and
lowering the travel speed of the autonomous mobile robot in response to the adjustment command.

19. The monitoring method for UV sterilization according to claim 18, further comprising:
notifying that there is the decayed lamp by generating a notification message.

20. The monitoring method for UV sterilization according to claim 11, further comprising:
receiving, by the autonomous mobile robot, a startup command from the monitoring platform, wherein enabling the autonomous mobile robot to move at the travel speed and driving the plurality of UV lamps of the autonomous mobile robot to emit the UV light are executed in response to the startup command;
receiving, by the autonomous mobile robot, a stop command from the monitoring platform; and
stopping driving the plurality of UV lamps in response to the stop command.
